(19) United States
(12) Patent Application Publication
(10) Pub. No.: US 2008/0050390 A1
(43) Pub. Date: Feb. 28, 2008

(54) Administration of gamma globulins to treat metastatic melanoma

(76) Inventors: Yehuda Shoenfeld, Ramat-Gan (IL); Miri Blank, Tel-Aviv (IL)

Correspondence Address: PEARL COHEN ZEDEK LATZER, LLP, 1500 BROADWAY 12TH FLOOR, NEW YORK, NY, 10036, US

(21) Appl. No.: 11/646,572

(22) Filed: Dec. 28, 2006

Related U.S. Application Data
(63) Continuation-in-part of application No. 11/606,364, filed on Nov. 30, 2006.
Application No. 11/606,364 is a continuation-in-part of application No. 09/405,050, filed on Sep. 27, 1999, now abandoned.
Application No. 09/405,050 is a continuation-in-part of application No. 08/487,803, filed on Jun. 7, 1995, now Pat. No. 5,965,130.
Application No. 08/487,803 is a continuation of application No. 08/340,094, filed on Nov. 15, 1994, now Pat. No. 5,562,902.
Application No. 08/340,094 is a continuation-in-part of application No. 08/212,361, filed on Mar. 14, 1994, now abandoned.

(51) Int. Cl. A61K 39/395; A61P 35/00

(52) U.S. Cl. 424/155.1; 424/174.1

(57) ABSTRACT

This invention relates to cancer therapy and in particular to the administration of gamma globulins to inhibit both primary tumor and metastasis and augment treatment of primary cancerous tumors. In accordance with this invention, the treatment of various cancerous diseases is accomplished by administering a preparation containing intact gamma globulins or fragments thereof.

### CROSS REFERENCE TO RELATED APPLICATIONS

This is a continuation-in-part of U.S. patent application Ser. No. 11/606,364 filed Nov. 30, 2006, which is a continuation-in-part of U.S. patent application Ser. No. 09/405,050, filed Sep. 27, 1999, now abandoned, which is a continuation-in-part of U.S. patent application Ser. No. 08/487,803, filed Jun. 7, 1995 now U.S. Pat. No. 5,965,130, which is a continuation of U.S. patent application Ser. No. 08/340,094, filed Nov. 15, 1994 now U.S. Pat. No. 5,562,902, which is a continuation-in-part of U.S. patent application Ser. No. 08/212,361, filed Mar. 14, 1994 now abandoned, the specifications of which are herein incorporated by reference in their entirety.

### FIELD OF THE INVENTION

The present invention relates to cancer therapy and in particular to the administration of gamma globulins (IVIG) to inhibit both primary tumors and metastases and augment treatment of primary cancerous tumors. In accordance with this invention, the treatment of various cancerous diseases is accomplished by administering a preparation containing intact gamma globulins or fragments thereof.

### BACKGROUND OF THE INVENTION

The formation of metastases of malignant tumors, initiated from a primary tumor at more or less remote locations of the body, is one of the most serious effects of cancer and one for which a -satisfactory treatment protocol is currently unavailable. Cancer tumor metastasis is responsible for most therapeutic failures when the disease is treated, as patients succumb to the multiple tumor growth.

The extent to which metastases occur vary with the individual type of tumor. Melanoma, lymphoma, breast cancer, lung cancer, colon cancer and prostate cancer are among the types of cancers that are particularly prone to metastasize. When metastasis takes place, the secondary tumors can form at a variety of sites in the body, with lungs, liver, brain and bone being the more common sites.

The currently available methods of cancer therapy such as surgical therapy, radiotherapy, chemotherapy and other immunobiological methods have either been unsuccessful in preventing metastasis or these methods give rise to serious and undesirable side effects.

At the time of this filing, cutaneous melanoma affects approximately 62,190 individuals annually in the United States. Melanoma is one of a few cancers with an increasing incidence. The five-year survival for patients with metastatic disease is less than 10% with a median survival of six months. The incidence of melanoma has increased dramatically over the past 20 years, and the prognosis of a newly-diagnosed melanoma patient is directly related to the depth of invasion (Breslow level). Five-year survival rates range from 100% for intra-epidermal lesions to 49% if the lesion penetrates into the subcutaneous fat. The prognosis worsens significantly in cases where tumor cells have migrated to lymph nodes and beyond.

Metastases frequently affect the liver, lungs, bones and brain. Wide surgical resections of the primary lesion and, in some cases, sentinel lymph node biopsy are the mainstays of therapy. Metastatic disease is treated by dacarbazine chemotherapy or IL-2, and about 10% of patients may achieve a durable complete response with the latter. Alpha interferon (IFN-α) adjuvant therapy is currently the standard of care for patients who have a high risk of relapse of metastatic disease. When distant metastatic disease has been established (stage IV) the prognosis is poor, with less than 10% of patients surviving at five years and a median survival of 6 months. In these cases chemotherapy with dacarbazine or immunotherapy with interleukin-2 are used. Up to 25% of these Stage IV patients may respond to interleukin-2, and 8-10% may achieve a durable complete response.

Despite the introduction of new combination chemotherapy regimens, immunotherapies, cancer vaccines and gene therapy during the past decade, no major improvements in survival have been achieved. Therefore, there exists a need for methods for inhibiting melanoma metastasis. In particular, methods which inhibit (micro)metastasis without causing serious side.

### SUMMARY OF THE INVENTION

In one embodiment, the invention provides a method of treating metastatic melanoma in a human subject comprising the step of administering to the subject a preparation of IVIG or fragments thereof, thereby preventing tumor cell proliferation, invasiveness, metastasis or their combination.

In another embodiment, provided herein, is the use of IVIG or fragments thereof in a composition for inhibiting metastatic melanoma in a human subject.

### BRIEF DESCRIPTION OF THE DRAWINGS

FIG. 1 shows patient survival since the start of treatment with IVIG (Kaplan-Meir survival curve)

### DETAILED DESCRIPTION OF THE INVENTION

### Definitions

As used herein, “gamma globulin” is the serum globulin fraction that is mainly composed of IgG antibody molecules.

As used herein, “IVIG” or “intravenous immunoglobulins” refers to gamma globulin preparations suitable for intravenous use, such as those IVIG preparations commercially available from several sources.

As used herein, “fragments” of IVIG or gamma globulin are portions of intact immunoglobulins such as Fc, Fab, Fab′, F(ab′)₂ and single chain immunoglobulins.

“Metastasis”, as used herein, is defined as the migration or transfer of malignant tumor cells, or neoplasms, via the circulatory or lymphatic systems or via natural body cavities, usually from the primary focus of tumor, cancer or a neoplasia to a distant site in the body, and the subsequent development of one or a plurality of secondary tumors or colonies thereof in the one new or the plurality of new locations. In another embodiment, “metastases” means the secondary tumors or colonies formed as a result of metastasis and encompasses micro-metastases.

As used herein, “inhibition of metastasis” is defined as preventing or reducing the development of metastases.

“Intracavitary administration”, as used herein, refers in one embodiment to administering a substance directly into a body cavity of a subject. Such body cavities include the peritoneal cavity, the pleural cavity and cavities within the central nervous system (intrathecal).

“Intravascular administration” as used herein, refers in one embodiment to administering a substance directly into circulatory system of a subject. The circulatory system is comprised of the the venous (veins) and arterial (arteries) systems.

The term “about” as used herein refers to quantitative terms plus or minus 5%, or in another an embodiment plus or minus 10%, or in yet another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

Gammaglobulins suitable for intravenous administration are commonly referred to as Intravenous Immunoglobulins (IVIG) and are commercially available from several sources. The commercially available IVIG preparations contain mainly IgG molecules. IVIG has been used in replacement therapy in primary immunodeficiency syndromes and in secondary immunodeficiencies as well as for the prevention and in the treatment of infectious diseases. Furthermore, IVIG has also been used for immune modulation of patients with autoimmune and immune-complex diseases (See, Shoenfeld Y, Katz U. IVIG therapy in autoimmunity and related disorders: our experience with a large cohort of patients. Autoimmunity 38: 123-137, 2005 and Shoenfeld Y, Krause I. IVIG for autoimmune, fibrosis, and malignant conditions: our experience with 200 patients. J Clin Immunol 24: 107-114, 2004).

According to a National Institutes of Health (NIH) Consensus Conference report, the incidence of adverse side effects associated with IVIG use in humans, used at dosage regimens comparable to the ones contemplated by the present invention, is usually less than 5% with most of those reactions being “mild and self-limited.” The report adds that “severe reactions occur very infrequently and usually do not contraindicate further IVIG therapy.” The NIH report also notes that “neither HIV nor hepatitis B infection has been transmitted to recipients of products currently licensed in the United States.” NIH Consensus Conference, “Intravenous Immunoglobulin: Prevention and Treatment of Disease”, JAMA, 264, pp. 3189-3193 (1990 ).

The present invention stems from our discovery that IVIG as a whole molecule or fragments thereof are extremely useful for the treatment of cancerous diseases.

The gamma globulin preparations that may be used according to the present invention include commercially available preparations of intact IVIG and preparations of the Fc, F(ab′)₂ fragments of IVIG or their combination. Recombinantly produced gamma globulins and their fragments may also be used according to this invention. The use of recombinant single chain antibodies is also envisioned.

In one embodiment, the gamma globulins may be prepared from the whole blood of one or more donors, preferably from a plurality of donors. In certain embodiments, the donors comprise mature members of the species so as to assure that each donor has been exposed to a number of different antigens during their lifetime and will thus have developed immunity against a variety of antigens. The use of a plurality of donors increases in another embodiment, the type and number of different gamma globulins obtained from the collected blood

The dosage of IVIG and the method of administration will vary with the severity and nature of the particular condition being treated, the duration of treatment, the adjunct therapy used, the age and physical condition of the subject of treatment and like factors within the specific knowledge and expertise of the treating physician. However, single dosages for intravenous and intracavitary administration can typically range from 50 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 50 mg/kg/day for 5 consecutive days per month or 2 g/kg/day once a month. The IVIG, according to the present invention, was found to be effective in inhibiting metastasis in animal models when administered by intravenous or intraperitoneal injection and in the dose range of 500-1000 mg/kg/week.

In another embodiment of this invention, the IVIG preparation is administered via the subcutaneous route. The typical dosage for subcutaneous administration can range from about 4 to 20 mg/kg body weight. The IVIG according to the present invention was found to be effective in inhibiting metastasis in mice when administered subcutaneously in the dose 200 g/mouse. According to the present invention IVIG may be administered as a pharmaceutical composition containing a pharmaceutically acceptable carrier. The carrier must be physiologically tolerable and must be compatible with the active ingredient. Suitable carriers include, sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

The intravenous immunoglobulins administered according to the present invention act as antimetastatic agents resulting in the reduction of tumor colony number as well as tumor colony size. They can also act prophylactically i.e., to prevent metastasis of tumors. The intravenous immunoglobulins according to this invention may also be used to reduce the size of the primary tumor.

The treatment described in the present invention may also be used either preceding, during or subsequent to a surgical procedure to remove the primary tumor. Frequently, metastasis of tumor cells will occur as a result of the physical manipulation of the tumor during surgery. However, the use of the treatment described in the present invention in conjunction with (i.e., before, during and/or after) surgery will reduce the risk of metastasis and consequently this combination of methods would be a more attractive treatment option for the complete elimination of cancerous tumors.

Similarly, other treatment modalities such as chemotherapy, radiation therapy and immunotherapy may also be used in conjunction with (i.e., before, at the same time as, or after) the methods of the present invention.

Although not wishing to be bound by any particular theory, intravenous immunoglobulins inhibit metastasis according to one or more embodiment of the following mechanisms.

It is known that tumor metastasis occurs following a detachment of single cancerous cells from the tumor, their migration to adjacent or distal tissues, and their seeding and homing in the new organ. The migration process takes place through adhesion molecules which enable the tumor cells to adhere to the blood vessel wall, to penetrate the blood stream and then to emerge and seed in another tissue. In one embodiment, when whole IVIG or the F(ab′)₂ fragments of IVIG are administered, they interfere with the binding of adhesion molecules responsible for the transmission of the tumor cell to and from the blood vessel, and thus prevent the dissemination of the tumor cells to other tissues in the body.

In another embodiment the presence of antibodies or anti-idiotypes in the IVIG mixture bind to the tumor cells and induce their lysis in the presence of complement or enhance entrapment of the tumor cells by Fc receptors on the reticuloendothelial system (RES).

The effect of IVIG on the dissemination of tumors according to the present invention is demonstrated by the following examples carried out in murine models of melanoma. Additionally, we also present clinical data of a representative human melanoma patient treated with IVIG. These examples are set forth so that this invention may be better understood and are not to be construed as limiting its scope in any manner.

In one embodiment, the methods and compositions of IVIG therapy described herein are effective and safe in the treatment of patients with metastatic melanoma who have failed to respond to standard therapy. Several mechanisms may be involved in generating the anti-tumor effects of IVIG used in the methods and uses described herein. These include in one embodiment, induction of interleukin-12 (IL-12) secretion that leads to natural-killer-cell activation, or inhibition of matrix metalloproteinase-9 (MMP-9) mRNA expression, suppression of tumor cell growth, inhibition of nuclear factor kappa B (NFκB) activation, degradation of inhibitor of kappa B (IκB), cell-cycle arrest at G1, or their combination in other embodiments. In another embodiment, adverse events associated with IVIG administration are mild and transient.

In one embodiment, the compositions and methods described herein are useful in the treatment of metastatic melanoma. According to this aspect of the invention and in one embodiment, provided herein is a method of treating metastatic melanoma in a human subject, comprising the step of administering to the subject a preparation of IVIG or fragments thereof, thereby preventing tumor cell proliferation, invasiveness, metastasis or their combination.

In one embodiment, the term “antibody” includes complete antibodies (e.g., bivalent IgG, pentavalent IgM) or fragments of antibodies which contain an antigen binding site in other embodiments. Such fragments include in one embodiment Fab, F(ab′)₂, Fv and single chain Fv (scFv) fragments. In one embodiment, such fragments may or may not include antibody constant domains. In another embodiment, Fab's lack constant domains which are required for Complement fixation. ScFvs are composed of an antibody variable light chain (V_{L}) linked to a variable heavy chain (V_{H}) by a flexible hinge. ScFvs are able to bind antigen and can be rapidly produced in bacteria or other systems. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

Antibodies of the invention can be monoclonal antibodies (mAb) in one embodiment, or polyclonal antibodies in another embodiment. Antibodies of the invention which are useful for the compositions, methods and kits of the invention can be from any source, and in addition may be chimeric. In one embodiment, sources of antibodies can be from a mouse, or a rat, a plant, or a human in other embodiments. Antibodies of the invention which are useful for the compositions, and methods of the invention have reduced antigenicity in humans (to reduce or eliminate the risk of formation of anti-human antibodies), and in another embodiment, are not antigenic in humans. Chimeric antibodies for use the invention contain in one embodiment, human amino acid sequences and include humanized antibodies which are non-human antibodies substituted with sequences of human origin to reduce or eliminate immunogenicity, but which retain the antigen binding characteristics of the non-human antibody.

In one embodiment, the preparation of IVIG or fragments thereof used in the methods and compositions described herein is administered intravenously, intracavitarily, intratumorally or subcutaneously, at a dosage of between about 0.1 g/kg/month to about 2 g/kg/month.

IVIG is administered in one embodiment, at an intermediate dose (1 g/kg of body weight per course), and infused at a rate in accordance with published recommendations.

In one embodiment, IVIG can be administered safely in heavily pretreated patients with advanced metastatic melanoma, even in the presence of exacerbating risk factors. Although some risk factors for thromboembolic events and renal failure are present (such as in cases of advanced age in some patients and a malignant condition which may be pro-thrombotic), patient immobilization, simultaneous enforcement of slow infusion rates of IVIG and good hydration, in certain embodiments, are important factors for the prevention serious adverse events attributable to the IVIG administration.

A person skilled in the art would readily recognize that the mode of administration, and the dosage will depend on many factors, such as inter-alia the stage of cancer of the subject, or the extent of metastases, as well as other factors in certain embodiments.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as liposomes, microemulsions, micelles, unilamellar or multilamellar vesicles, erythrocyte ghosts, virosomes, or spheroplasts, antibodies or cell specific ligands. Targeting may be desirable in one embodiment, for a variety of reasons, e.g. if the agent is unacceptably toxic, or if it would otherwise require too high a dosage, to achieve a substantially higher local concentration, or if it would not otherwise be able to enter the target cells.

In one embodiment, the metastatic melanoma treated by the methods and compositions described herein, is metastasizable to an organ selected from the group of lymphatic system, lung, liver, brain or bone.

In one embodiment, metastases are the leading cause of treatment failure and death of patients affected by metastatic melanoma, which makes them a major therapeutic target. The metastatic process comprises in another embodiment, a series of complex interactions between cancerous cells and host cells or tissues. A cell originating from a melanoma site must undergo several modifications to become metastatic. These include loss of adhesion with surrounding cells in one embodiment, or migration towards vessels, destruction of the basement membrane, passage in the blood stream and escape from the immune system, or their combination in other embodiments. The cells must then arrest and extravasate into the target tissue, and growth in this tissue where a neoangiogenesis leads to its blood supply. Accordingly, and in another embodiment, the methods and compositions provided herein are effective in the treatment of metastases of melanoma by interfering with the metastatic processes in a subject.

In another embodiment, the methods and compositions provided herein, are effective in the prevention or inhibition of proliferation, invasion, metastatic capacity or their combination, of metastatic melanoma cells. In yet another embodiment, described herein is the use of IVIG or fragments thereof in a composition for inhibiting metastatic melanoma in a human subject, whereby the IVIG fragment is F(ab′)₂, Fc or a combination thereof.

The following examples are presented in order to more fully illustrate the preferred embodiments of the invention. They should in no way be construed, however, as limiting the broad scope of the invention.

### EXAMPLES

Materials and Methods:

Tumor Cells

One Tumor cell line from murine origin were used: B16-F10 melanoma cells is (purchased from American Type Tissue Culture Collection, Rockville, Md.). The cells were routinely maintained in RPMI medium containing 10% fetal calf serum. Twice a week the cells were transferred to a freshly prepared medium

Experimental Animal Models

2-3 months old C57BL/6J mice were used during the study. To examine the efficacy of gamma globulin in vivo, 2 types of solid tumors were induced in C57BL/6J mice, e.g. sarcoma (MCA-105) and melanoma (B16-F10). The tumor cells were induced either by intravenous (IV) injection which led to their seeding and lodging in the lung or by intraperitoneal (IP) introduction where the cancerous cells developed local lesions in the peritoneum. Some of the mice were sacrificed following 3-5 weeks and examined for metastatic foci in the lungs or spread of tumors in the peritoneum. In another group of mice, survival time was observed. Mice that were injected with tumor cells by IV mode were treated by IV infusion of gamma globulin, whereas in animals in whom the tumor was induced directly in the peritoneum, the gamma globulin preparation was administered through IP injection.

Gamma Globulin Preparations

Gamma Globulin Preparations: Human gamma globulin suitable for intravenous use (IVIG) was obtained from Miles Inc. (Biological Products Division, West Haven, Conn.). A 5% solution (5 gr in 100 ml diluent; Catalogue No. 0640-12) was used for all experiments.

Unless otherwise indicated, the volume of IVIG inoculated was 500 μl. per animal on each treatment which amounted to 25 mg of gamma globulin per animal. Other preparations used were a whole molecule human gamma globulin or an F(ab′)₂ fragment both purchased from Jackson Immunoresearch Laboratories, Inc., West Grove, Pa., (Code Numbers 009-000-003 and 009-000-006 respectively). These latter preparations are different from the one obtained from Miles Inc. in that they are prepared from a donor pool of 30 whereas the Miles preparation is from a donor pool of 3000 or more individuals.

### Example 1

### Effect of Gamma Globulin on the Development of Metastatic Melanoma in C57BL/6J Mice

An experimental model for metastatic melanoma was established using the B16-F10 mouse melanoma cell line. The induction of the melanoma was carried out by IV injection of the tumor melanoma cells which are subsequently seeded in the mice lung and form black metastatic foci. Approximately 24 days following tumor inoculation, the mice die.

In the present experiment the mice were injected either with 2×10⁵ tumor cells or with 5×10⁵ cells and were treated intravenously with IVIG (Miles). The mice were sacrificed on day 18 and the efficacy of the treatment was determined by counting the number of the black metastatic foci in the lungs of the animals.

A. Inoculation of Mice with 2×10⁵ B16-F10 Melanoma Cells.

20 mice were IV injected with 2×10⁵ melanoma cells and were divided into 4 groups: (a) Control group, mice inoculated with tumor cells only; (b) The mice treated with one IV injection of IVIG on day 0 (the day of tumor administration); (c) The mice treated 2 times, on day 0 and on day 4; and (d) The mice treated 3 times on days 0, 4 and 9.

The mice were sacrificed on day 18 and the number of the metastatic foci in the lungs was evaluated. Table I summarizes the results. One treatment reduced the number of metastatic foci by 80%, while no foci could be detected following two or three treatments.

TABLE I

| GROUP | NO. OF FOCI |
|---|---|
| Control | 20 ± 4 |
| 1 Treatment | 4 ± 2 |
| 2 Treatments | 0 |
| 3 Treatments | 0 |

Black metastatic foci are seen in the control group, less foci in the group that was treated with one injection of IVIG and none is seen in the lungs that were derived from mice treated by two or three injections of IVIG.

B. Inoculation of Mice with 5'10⁵ B16-F10 Melanoma Cells

In order to explore whether IVIG is capable of preventing metastasis when a larger mass of melanoma was involved, the following experiment was conducted. Mice were injected with an increased number of tumor cells (5×10⁵) and divided into 2 groups: (a) Control group, inoculated with tumor cells only; and (b) mice treated with 2 IV injections of WVIG on day 0 and on day 8 following tumor inoculations.

On day 18, the mice were sacrificed. Evaluation of the lung metastatic foci revealed a marked decrease in their number in the IVIG treated group. The results are summarized in Table II.

TABLE II
Reduction in number of metastatic foci in the lungs of mice injected with
5 × 10⁵ B-16 F10 melanoma cells and then treated with IVIG

| GROUP | NO. OF FOCI |
|---|---|
| Control | 165 ± 13.4 |
| Two Treatments | 16.3 ± 3.9 |

There is about a 90% reduction in the number of foci in the treated mice when compared to the control group. These results show that WVIG is capable of inhibiting metastatic spread of melanoma even when a larger tumor mass is involved.

C. Effect of Gamma Globulin on the Survival of Melanoma Bearing Mice

4×10⁵ B16-F10 cells were injected IV to C57BL/6J mice. The mice were treated from day 0 and every 7^{th} day thereafter with 500 μl Miles IVIG. 24 days following the inoculation of the tumor, the mice from the control group began to die. While on day twenty six 100% of the control mice were dead, 100% of the IVIG treated mice were alive. On day 40, of the treated mice were still alive.

### Example 2

### Comparison of the Effect of Intact Gamma globulin and F(ab′)₂ Fragment on the Development of B-16 Melanoma in C57BL/6J Mice

Mice were IV inoculated with 2.5×10⁵ B16-F10 melanoma cells. The mice were divided into 3 groups:
a) A control group;
b) mice treated with whole molecule IVIG (Jackson Immunoresearch Laboratories Inc.); 5 mg in a volume of 330 μl was injected IV on days 0, 3, 7 and 12; and
c) a group of mice that was treated with 5 mg of F(ab′)₂ fragment of IVIG (Jackson) in a volume of 500 μl on the same days as in (b).

The mice were sacrificed on day 17 and black metastatic foci were counted in the lung. In the control group 160±18 metastatic foci were counted in comparison to 68±12 in the group treated with the preparation of intact IVIG, and 13±4 foci in the mice treated with the preparation of F(ab′)₂ fragments of IVIG. This result indicates that both intact IVIG and their F(ab′)₂ fragments are effective in inhibiting metastases. The observed difference in the effectiveness between the intact IVIG and the F(ab′)₂ preparation is probably due to the difference in the specific activities of the two solutions used. The difference between the results of Example 1 where the mean number of metastatic foci was 4 when intact IVIG were administered (Table I) and the present example where the mean number of foci was 68 is probably due to the fact that in Example 1, 25 mg of whole IVIG was injected on day 0, whereas in this example only 5 mg was injected on day 0.

### Example 3

### Effect of Gamma Globulin in the Inhibition of Metastasis of Melanoma Following Surgical Removal of Primary Tumor

C57BL6J mice were injected in the foot pad with 2.5×10³ melanoma cells. After 21 days the leg in which tumor developed was amputated. On the same day the mice were divided into two groups, one group was treated by intravenous injection of 25 mg IVIG (Sandoz Pharma. Ltd., Basle, Switzerland; Lot-4.372.256.0) and the other group with phosphate buffered saline (PBS). Ten days later, the mice were examined for signs of tumor development. 60% of the control group developed tumor with a mean size of 3±0.8 cm. Those mice died during the first month following amputation.

In the IVIG treated group, only 14% of the mice developed tumor (mean size 2.7±1.2 cm). These mice also died during the first month following amputation. The remaining 86% of the IVIG treated mice did not develop tumor and were still alive 45 days following surgery.

### Example 4

### Effect of Low Dose, Subcutaneous Administration of Gamma Globulin in the Inhibition of Melanoma Metastasis

C57BL6J mice were injected intravenously with 2.5×10⁵ B16 melanoma cells per mouse. Immediately thereafter, the mice were administered IVIG preparations via the subcutaneous route in the chest area. Four groups of mice (20 mice/group) received 200 g/mouse of one of the following commercially available IVIG preparations obtained from Baxter (Gammagard S/D 2.; Lot-93H23AB12C), Isiven (Isiven V. I. 2. lot-IS238C6193V), Miles (Gamimmune N 5%; Lot-640N023) and Sandoz (Lot-4. 372.256.0). A fifth group of mice receiving intravenous PBS administration acted as the control group. The mice were sacrificed 18 days later and their lungs examined for the presence of metastatic foci. The following table depicts the results.

TABLE III

| GROUP | NO. OF FOCI | % INHIBITION |
|---|---|---|
| Control | 50 | — |
| Baxter | 18.6 | 62.8 |
| Miles | 28.5 | 43.0 |
| Isiven | 18.1 | 63.8 |
| Sandoz | 27.9 | 44.2 |

As shown above, low dose, subcutaneous IVIG administration inhibited melanoma metastasis by an average of 53.45% when compared to mice treated with PBS.

### Example 5

### Effect of Intravenous Administration of Gamma Globulin to a Representative Melanoma Patient

A forty-two year old male underwent surgery in September 1989 for the wide excision of a malignant melanoma lesion (depth 1.3 mm) on his left thigh. In May 1991, he underwent a hyperthermic perfusion of the leg with cisplatinum because of a local recurrence of the melanoma in a left femoral lymph node. At that time there was no evidence of metastatic disease in the patient. In February 1993, computerized tomography (CT) scans of the chest and abdomen revealed mass lesions in the spleen (one lesion), the liver (five lesions, the largest being 3×3 cm) and the lungs (four lesions, the largest being 1.5×1.5 cm). Despite the lesions, the patient was asymptomatic. Soon thereafter, a treatment with IVIG was begun. The patient was intravenously administered IVIG (Miles) at a dose of 400 mg/kg/day for 5 consecutive days per month. After five cycles of treatment, all of the spleen and liver metastases disappeared and there was also a slight reduction in the lung lesions. Afterwards, the patient's condition deteriorated with the appearance of new bone and subcutaneous lesions. He continued to receive IVIG with minor reemergence of liver metastases. The patient expired after receiving 12 cycles of IVIG.

As will be appreciated by one of skill in the art, large liver and spleen metastases of melanoma do not regress spontaneously. Furthermore, it is well known that after the detection of such large metastases, the survival time of patients is usually no longer than a few months.

### Example 7

### Effect of Intravenous Administration of Gamma Globulin to Nine Refractive Melanoma Patients

Materials and Methods

Patients

Nine adult patients with non-resectable metastatic melanoma who failed prior chemotherapy and immunotherapy were enrolled in the study. Enrollment criteria included: at least one measurable site of disease, Eastern Cooperative Oncology Group (ECOG) performance status (PS) between 0-2, serum creatinine level <2.0 mg/dl or creatinine clearance of at least 50 ml/min, and a life expectancy of at least three months. Patients with melanoma that metastasized to the central nervous system and those who underwent chemotherapy in the last four weeks were excluded, as were those with IgA deficiency.

IVIG Administration

VIGAM® LIQUID, was obtained containing 5% (w/v) Human Immunoglobulin and is manufactured by BioProducts Laboratory (BPL, Dagger Lane, Elstree, Herts. WD6 3BX, United Kingdom, batch numbers VLCN6797, VLAN6790, VLCN7050, VLAN7069). This IVIG preparation contains sucrose as a stabilizer (0.5 g sucrose/g immunoglobulin). IVIG was administered at 1 g/kg of body weight/cycle over a one to three day period at a maximum dose of 25 g per day; the infusion period that lasted for up to eight hours. Prior to the first IVIG course, 200 mg of hydrocortisone were administered by intravenous push injection. Concomitant use of additional systemic glucocorticoids, hormonal therapy or progestin (Megace, Provera), non-steroidal anti-inflammatory drugs (conventional or cyclooxygenase-2 inhibitors), granulocyte colony stimulating factor (G-CSF), erythropoietin (EPO) and alternative medicines were not permitted.

Treatment Courses and Patient Follow-up

Patients were scheduled for six courses of IVIG infusions to be given every 3 weeks. Before each course of IVIG, blood was taken for biochemical and hematological measurements, and the patients underwent a physical examination. Adverse events (AE) were monitored and recorded during and after IVIG infusion as well as between treatment cycles. One patient with stable disease after having completed the protocol was continued on IVIG therapy for an additional six courses (total of 12 IVIG cycles).

After the 3^{rd} and 6^{th} courses, a computerized tomographic scan (CT) was performed to allow assessment of the Response Evaluation Criteria in Solid Tumors (RECIST) score. Patients with progressive disease at any point were withdrawn from the study.

Efficacy Measurements

Efficacy was assessed using the RECIST Score. Once progressive disease was observed, time to tumor progression (TTP—the time from randomization until objective tumor progression) was calculated. The Eastern Cooperative Oncology Group (ECOG) performance status scale was recorded for each treatment cycle.

Adverse Events

Adverse events were recorded as reported by the patient and/or observed by the attending medical staff. Assessments were also made based on vital signs, as well as on changes of laboratory parameters (including hematology, chemistry).

Results

Patients

Five male and four female, patients with metastatic melanoma who had been previously heavily treated with anti-melanoma therapies were recruited into the study. Their ages ranged from 31 to 89 years old (mean 57 years; Table 1)

TABLE 1
Patient Demographics

| Patient Number | Gender | DOB | Pre Existing Conditions | LN Mets | Mets | Prev RX | ECOG PS |
|---|---|---|---|---|---|---|---|
| 1 | Female | 1957 | Brain mets excised | No | Spleen | Radiation | 1 |
| | | | | | Soft Tissue | Decarbazine | |
| | | | | | | Fotemustine | |
| | | | | | | INFa | |
| 2 | Female | 1916 | Diabetes mellitus | Yes | Pulmonary | Radiation | 2 |
| | | | Hypothyroidism | | Soft Tissue | Decarbazine | |
| | | | | | | Fotemustine | |
| | | | | | | INFa | |
| 3 | Female | 1945 | Anemia | Yes | Pulmonary | Radiation | 1 |
| | | | | | Soft Tissue | Decarbazine | |
| | | | | | | Cisplatin | |
| | | | | | | IL2 | |
| | | | | | | INF-a | |
| 4 | Male | 1949 | Diabetes mellitus | Yes | Liver | Radiation | 1 |
| | | | BPH | | Spleen | Decarbazine | |
| | | | | | Pulmonary | Cisplatin | |
| | | | | | Adrenal | IL2 | |
| | | | | | | INF-a | |
| 5 | Male | 1942 | NA | Yes | Liver | Radiation | 1 |
| | | | | | Pulmonary | Decarbazine | |
| | | | | | Pelvic LN | Cisplatin | |
| | | | | | | IL2 | |
| | | | | | | INF-a | |
| 6 | Male | 1946 | GERD | Yes | Liver | Radiation | 0 |
| | | | | | Pulmonary | Decarbazine | |
| | | | | | Peritoneal | Cisplatin | |
| | | | | | | IL2 | |
| | | | | | | INF-a | |
| 7 | Female | 1950 | Hepatitis C | Yes | Lung | Radiation | 2 |
| | | | Asthma | | Soft Tissue | Decarbazine | |
| | | | | | Retroperitoneal | Cisplatin | |
| | | | | | | Temzolomide | |
| | | | | | | IL2 (intralesional) | |
| | | | | | | INF-a | |
| 8 | Male | 1947 | Hypertension | Yes | Liver | Radiation | 1 |
| | | | | | Pulmonary | Decarbazine | |
| | | | | | Pelvic & inguinal nodes | Cisplatin | |
| | | | | | | IL2 | |
| | | | | | | INF-a | |
| 9 | Male | 1974 | NA | Yes | Bone | Radiation | 0 |
| | | | | | Liver | Vaccination | |
| | | | | | | Decarbazine | |
| | | | | | | Cisplatin | |
| | | | | | | IL2 | |
| | | | | | | INF-α | |

Safety Analysis

The occurrence of adverse events (AE) is detailed in Table 2. No grade severe toxicity was observed in any patient. There were no occurrences of renal failure or thromboembolic phenomena. One patient (No. 4) died with progressive cervical spinal cord compression some days after the first IVIG infusion. There were also three instances of chest pain noted (patients 4, 5, and 6) with only circumstantial linkage to the IVIG infusion, and there was no evidence of myocardial ischemia by electrocardiogram or cardiac enzyme measurements in any of these patients. Two patients developed skin rash and two had non-hemolytic anemia after IVIG therapy.

TABLE 2
Adverse events (AE) during treatment with IVIg

| | No. Patients |
|---|---|
| Symptom | with AE |
| Chest pain | 3 |
| Headache | 2 |
| Weakness/fatigue | 2 |
| Non-hemolytic anemia | 2 |
| Dermal rash | 2 |
| Hyponatremia | 1 |
| Hypertension | 1 |
| Diarrhea | 1 |
| Abdominal pain | 1 |

Efficacy Analysis

Tumor response rates as assessed by RECIST criteria are shown in Table 3. Two patients achieved stable disease. Time to tumor progression in patient 5 was eight months and in patient 1, three months. All other patients had a progressive disease. In patients with progressive disease, the survival spanned between 1-6 months.

TABLE 3
Tumor response in study patients

| Response | No. Patients |
|---|---|
| Complete response | 0 |
| Partial response | 0 |
| Stable disease | 2 |
| Progressive disease | 6 |
| Not applicable* | 1 |

*This patient died some days after the first IVIG dose due to cervical spinal cord compression, unrelated to IVIG, and could not be evaluated with the RECIST score.

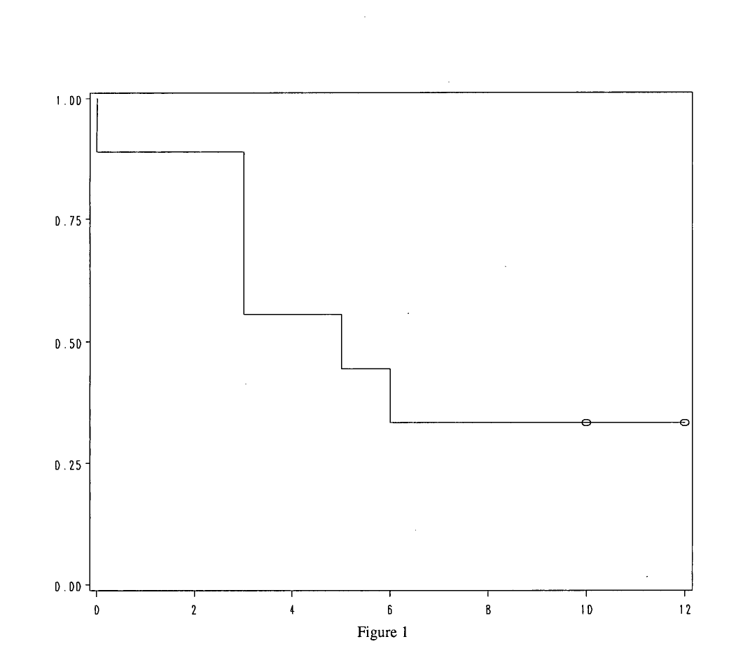

Both patients (Nos. 1 & 5) in whom stable disease was achieved are still alive 11 and 4 months after completing IVIG therapy (FIG. 1). One of these two patients, (No. 5), continued to receive IVIG beyond the six cycles (4.2 months) of this trial. IVIG continued to be administered according to the protocol, and his disease remained stable during 12 cycles (8.4 months) of therapy. This patient also did not deteriorate in his ECOG performance status; instead, the patient returned to work and led a normal life for a sustained period of time. It is noteworthy that overall, in this study, 75% of patients had stable ECOG performance status during the course of therapy despite objective evidence of tumor progression.

While we have described above specific examples of this invention, it will be apparent to those skilled in the field of cancer therapy that our basic methods may be altered according to need. Therefore, it will be appreciated that the scope of our invention is to be defined by the claims appended hereto rather than by the specific embodiments presented hereinbefore by way of example.

What is claimed is:

1. A method of treating metastatic melanoma in a human subject comprising the step of administering to the subject a preparation of IVIG or fragments thereof, thereby preventing tumor cell proliferation, invasiveness, metastasis or their combination.

2. The method of claim 1, whereby the preparation of IVIG, or fragments thereof is administered intravascularly, intracavitarily, intratumorally, intrathecally or subcutaneously.

3. The method of claim 2, whereby the preparation of IVIG is administered at a dosage of between about 0.05 g/kg body weight to about 2 g/kg body weight

4. The method of claim 1, whereby the metastatic melanoma has metastasized to lymphatic system, lungs, liver, brain, bone or a combination thereof.

5. The method of claim 2, whereby the subject is administered at least one other treatment modality, prior to, during or after the administration of the IVIG preparation.

6. The method of claim 2, whereby the subject is refractive to immunotherapy, chemotherapy or both.

7. The method according to claim 5, whereby the other treatment modality is chemotherapy, immunotherapy, radiation therapy, surgery or a combination thereof.

8. Use of WVIG or fragments thereof in a composition for inhibiting metastatic melanoma in a human subject.

9. The use of claim 8 whereby the IVIG fragment is F(ab′)₂, Fc or a combination thereof.
